(19) United States
(12) Patent Application Publication
(10) Pub. No.: US 2021/0059858 A1
(43) Pub. Date: Mar. 4, 2021

(54) METHODS FOR PREVENTING AND TREATING OCULAR DISEASE

(71) Applicants: University of Wisconsin-Milwaukee Research Foundation, Inc., Milwaukee, WI (US); LumiThera, Inc., Poulsbo, WA (US)

(72) Inventors: Janis T. Eells, Milwaukee, WI (US); Clark E. Tedford, Poulsbo, WA (US)

(21) Appl. No.: 17/013,484

(22) Filed: Sep. 4, 2020

Related U.S. Application Data
(60) Provisional application No. 62/895,936, filed on Sep. 4, 2019.

(51) Int. Cl. A61F 9/008; A61F 9/007

(52) U.S. Cl. CPC A61F 9/008; A61F 9/0079; A61F 2009/00885; A61F 2009/00851; A61F 2009/00848; A61F 2009/00863

(57) ABSTRACT

Methods for preventing and/or treating ocular diseases such as dry age-related macular degeneration, and/or improving or maintaining a retinal function, include administering non-invasive photobiomodulation light therapy in the context of a prior PBM treatment, on the basis of a patient pre-treatment characteristic. In some embodiments, methods comprise administering PBM therapy to subjects prior to onset of, or in early stages of, dry age-related macular degeneration.

### BACKGROUND

Age-related macular degeneration (AMD) is a retinal disease that results in irreversible, severe loss of vision, including legal blindness. Disease progression inevitably leads to significant visual dysfunction and serious compromises in quality of life (QoL). The prevalence of AMD is projected to affect 196 million by the year 2020 with expected growth to 288 million in 2040 (see Wong et al., Lancet Glob. Health 2(2):e106-e116 (2014)).

Progression of AMD is characterized by accumulation of membranous debrids, lipofuscin and extracellular material and complement deposition. The advanced late stage dry form of AMD, which accounts for 80-90% of cases, is characterized by retinal pigment epithelium (RPE) and outer retinal atrophy, whereas only 10-20% of AMD patients develop the exudative, wet late stage form, with choroidal neovascularization (CNV) as a hallmark of respective disease.

Current modalities for treating wet AMD include periodic intravitreal injections of anti-VEGF (Vascular Endothelial Growth Factor) compounds. Treatment options for the more frequent dry form of AMD have typically been limited to lifestyle changes and the use of vitamin supplements, demonstrating a significant unmet need for prophylactic and treatment plans for an expanding population base.

Accordingly, new modalities for preventing and treating dry AMD are needed. The presently disclosed embodiments address these needs and provide other related advantages.

### BRIEF DESCRIPTION OF THE DRAWINGS

FIG. 1 shows a schematic illustration of photoreceptor and bipolar ocular cells (left) and an exemplary electroretinogram (ERG) curve indicating signal outputs from these cells following light stimulus (right).

FIGS. 2A and 2B show Scotopic Intensity-Response Series in Nrf2^{-/-} mice following 12 weeks of treatment with 670 nm PBM or sham treatment, as described in the Examples. ERG alpha-wave (a-wave) (2A) and ERG beta-wave (b-wave) (2B) responses were measured in PBM-treated mice and sham-treated mice.

FIGS. 3A and 3B show Scotopic Intensity-Response Series in Nrf2^{-/-} mice following 12 weeks of treatment with 830 nm PBM or sham treatment. Differences in ERG a-wave (3A) and b-wave (3B) responses in PBM-treated mice versus sham-treated mice did not reach statistical significance.

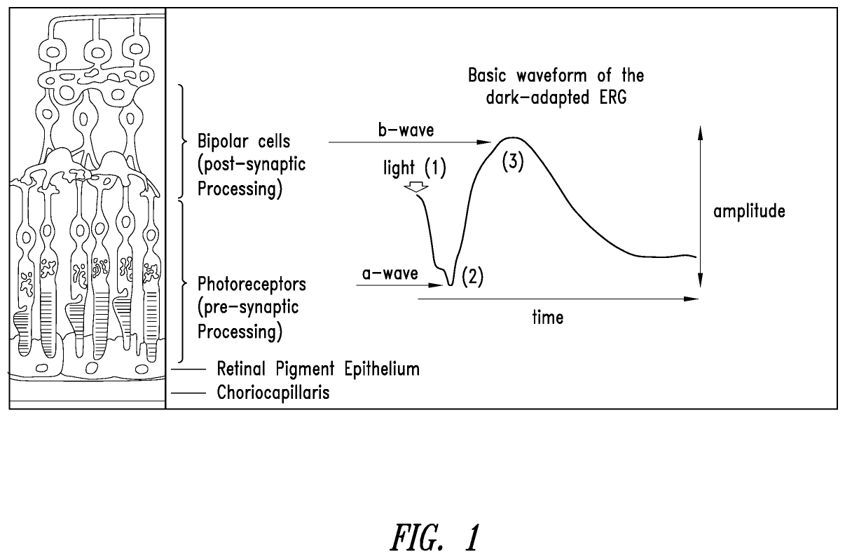

### DETAILED DESCRIPTION

In certain aspects, the present disclosure provides methods for preventing or delaying onset and/or progression of dry age-related macular degeneration (dry AMD), wherein the methods comprise use of photobiomodulation therapy (PBM) comprising light having a wavelength in a red and/or having a wavelength in a near-infrared wavelength range.

In certain aspects, methods are provided for preserving or improving a retinal function, such as a b-wave response to light stimulus (e.g., improving, increasing, extending, or preserving the b-wave response (e.g., intensity, duration, or both) to the light stimulus), an a-wave response to light stimulus (e.g., improving, increasing, or preserving extending, of the a-wave response (e.g., intensity, duration, or both) to the light stimulus), or both, in an eye of a subject that is at risk for developing or has dry AMD, preferably early-stage dry AMD, wherein the methods comprise use of PBM comprising light having a wavelength in a red and/or in a near-infrared wavelength range.

In certain embodiments, a subject to receive PBM according to the presently disclosed methods has a phenotype, symptom, or indicia that is characteristic of early-stage dry AMD or has a phenotype that is characteristic of an eye that is at risk of developing dry AMD.

In the present disclosure, it was surprisingly determined that PBM therapy comprising light in a red wavelength range and/or in a near infrared wavelength range protects against loss of retinal function in an art-accepted animal model of age-related retinal degeneration, including dry AMD. Notable benefits were observed when animals were treated with PBM prior to, or during an early stage or stages of, development or progression of retinal degeneration comprising one or more dry AMD-like phenotype.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as size or thickness, or length of time (e.g., seconds, minutes, hours, days, weeks, months) is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

The term “consisting essentially of” is not equivalent to “comprising”, and refers to the specified materials or steps, or to those that do not materially affect the basic characteristics of a claimed invention.

The terms “treat” and “treatment” refer to medical management of a disease, disorder, or condition of a subject (i.e., patient, host, who may be a human or non-human animal) (see, e.g., Stedman's Medical Dictionary). In general, an appropriate dose and treatment regimen according to the methods and compositions described herein results in a therapeutic or prophylactic benefit. Therapeutic or prophylactic benefit resulting from therapeutic treatment or prophylactic or preventative methods include, for example, an improved clinical outcome, wherein the object is to prevent delay, or retard or otherwise reduce or limit (e.g., decrease in a statistically significant manner relative to an untreated control) an undesired physiological change or disorder, or to prevent, delay, retard or otherwise reduce or limit the expansion or severity of such a disease or disorder. Beneficial or desired clinical results from treating a subject include attenuation, abatement, lessening, or alleviation of symptoms that result from or are associated with the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease onset and/or progression; amelioration or palliation of the disease state; and remission (whether partial or total), whether detectable or undetectable; or overall survival.

“Effective amount” or “therapeutically effective amount” refers to an amount of a composition, combination, or PBM therapy which, when administered to a mammal (e.g., human), is sufficient to aid in preventing or treating a disease (or onset or progression thereof). The amount of PBM therapy that constitutes an effective amount will vary depending on the condition to be treated and its severity, the manner of administration, and the age of the mammal to be treated, but can be determined routinely by one of ordinary skill in the art having regard to her own knowledge and to this disclosure. Where the intended treatment is prophylaxis, the terms “therapeutically effective” and “prophylactically effective” and “effective” may be used interchangeably. When referring to an individual active component (e.g., light of a PBM wavelength or wavelengths), administered alone, an effective dose refers to that component alone. When referring to a combination, an effective dose refers to combined amounts of the active components (e.g., light of different wavelengths) that result in the therapeutic effect, whether administered serially, concurrently or simultaneously. Exemplary effective amounts of PBM for prophylaxis or treatment of dry AMD are provided herein.

As used herein, “administration” of a composition or therapy refers to delivering the same to a subject, regardless of the route or mode of delivery. Administration may be effected a single time, continuously (i.e., without stopping, or at regular intervals without a predetermined end), or intermittently. Administration may be for treating a subject already confirmed as having a recognized condition, disease or disease state, or for treating a subject susceptible to or at risk of developing such a condition, disease or disease state. Methods, devices, and parameters for administering PBM include those provided herein.

Subjects in need of the methods described herein include those who already have the disease or disorder, as well as subjects prone to have, or at risk of developing, the disease or disorder. Subjects in need of prophylactic treatment include subjects in whom the disease, condition, or disorder is to be prevented (i.e., decreasing the likelihood of occurrence, or recurrence, of the disease or disorder, or altogether preventing occurrence or recurrence). Clinical benefits provided by the methods described herein can be evaluated using in vitro assays, preclinical studies, and clinical studies in subjects to whom administration of the compositions is intended to benefit, as described in the examples.

Prevention or treatment of an ocular disorder (e.g., dry AMD) in an eye or eyes can comprise an effect determinable or measurable using any of a number of metrics, including, for example: an improvement in retinal function characterized, for example, by an increased amplitude (e.g., a statistically significant increase in amplitude as compared to the same eye prior to receiving PBM therapy, or as compared to a reference eye that did not receive PBM therapy according to the disclosure)) and/or duration of an electroretinogram (ERG) alpha wave and/or an ERG beta wave in response to a flash (e.g., a flash having an intensity of from 100 mcd·s/m² to 25,000 mcd·s/m²), as compared to a reference amplitude and/or duration of response (e.g., a response amplitude of the at-risk or diseased eye or eyes of the same subject prior to treatment, or a clinically accepted response amplitude and/or duration characteristic of an AMD disease state or of an eye that is at-risk for onset or progression of AMD); a statistically significant improvement in a best corrected visual acuity (BCVA) letter score according to an optometry chart (e.g., an ETDRS chart (Precision Vision, USA)) or a Snellen equivalent thereof; a statistically significant improvement in contrast sensitivity (CS) using, for example, the Functional Acuity Contrast Test (FACT), which can be performed using a chart that includes a series of grating patches with functionally different spatial frequencies; a statistically significant improvement in retinal sensitivity (RS) or fixation stability, e.g., as determined by microperimetry; a statistically significant decrease in the rate of growth, absolute growth, volume, thickness, number, or geographic spread of drusen (e.g., measurable using ocular coherence tomography such as SD-OCT, such as with a Spectralis OCT or TruTrack™ device (Heidelberg Engineering, Heidelberg, Germany)); or a statistically significant improvement in responses to one or more questions from the National Eye Institute VFQ-25 Questionnaire (e.g., Questions from Part II: Difficulty with activities; e.g., Q5-Q14). VFQ25 can be found online at, for example, https://nei.nih.gov/sites/default/files/nei-pdfs/vfq_sa.pdf). It will be understood that a statistically significant response or improvement can be in response to any functional variant of a herein-described test (e.g., a question in a questionnaire can be worded differently than the specific language used in the VFQ-25, but is still within the scope of the present disclosure when it is substantively the same as a reference question in VFQ-25).

It will also be understood that an increase or decrease in a function or risk factor or disease marker of an eye can be determined by reference to the same eye (or a different eye) of the subject prior to receiving therapy, and/or to an eye of a reference subject.

It will be understood that a reference subject can be: (i) a subject of a same or a similar: age or age group; gender; ethnic group; diagnosis or lack of diagnosis of AMD; smoking history; overweight or obese status; historical sun exposure; presence of one or more AMD phenotypes; family history regarding ocular disease and/or hyperglycemia and/or diabetes; and/or general health as the subject administered PBM according to a currently disclosed method; and/or (ii) a typical or average subject within a population (e.g., local, regional, or national), including within a population defined according to: age or age group; gender; ethnic group; diagnosis or lack of diagnosis of AMD; smoking history; overweight or obese status; historical sun exposure; presence of one or more AMD phenotypes; family history regarding ocular disease and/or hyperglycemia and/or diabetes; and/or general health. A reference subject does not receive PBM according to the present disclosure, and, in certain embodiments, does not receive prophylaxis or treatment for AMD.

In certain embodiments, preventing or delaying onset and/or progression of dry AMD comprises slowing, stopping, preventing, and/or reversing one or more of the following phenotypes or symptoms: (a) hypo-pigmentation of the retinal pigment epithelium (RPE); (b) hyper-pigmentation of the RPE; (c) mottling of the RPE; (d) vacuolation in the RPE and/or the sub-RPE space, optionally in the Bruch's membrane; (e) multivesicular bodies in the RPE and/or the sub-RPE space, optionally in the Bruch's membrane; (f) a region of partial or complete loss of RPE; (g) the presence of drusen or drusen-like deposits, optionally being dome-shaped and/or having hard borders and/or a whitish color; (h) accumulation of lipofuscin; (i) spontaneous choroidal neovascularization (CNV); (j) subretinal hemorrhage; (k) subretinal cellular infiltrates; (m) basal laminar deposits in the RPE; (n) a region of thickening of Bruch's membrane; (o) vision loss in the eye, wherein vision loss optionally comprises a blurred spot in a center of a field of vision; (p) medium-size drusen, large-size drusen, soft drusen, drusen having a soft border, and/or drusen having a yellow color; (q) geographic atrophy (GA) affecting a fovea; (r) difficulty seeing in a center of a field of vision; (s) difficulty seeing in dim light; (t) difficulty seeing and/or perceiving straight lines; (u) difficulty seeing and/or perceiving colors; (v) an ERG response amplitude of a b-wave that is reduced (e.g., lower and/or less sustained) as compared to a response amplitude of a b-wave in a healthy eye (in certain embodiments, a b-wave amplitude of an at-risk or diseased eye is about 200 μV, about 150 μV, or less to a flash of an intensity that is about 25,000 mcs·s/m², 20,000 mcs·s/m², 15,000 mcs·s/m², 10,000 mcs·s/m², 5,000 mcs·s/m², or less); and (w) an ERG response amplitude of an a-wave that is reduced (e.g., lower and/or less sustained) as compared to a response of an a-wave in a healthy eye (in certain embodiments, an a-wave amplitude of an at-risk or diseased eye is about 150 μV, about 100 μV, or less to a flash of an intensity that is about 25,000 mcs·s/m², 20,000 mcs·s/m², 15,000 mcs·s/m², 10,000 mcs·s/m², 5,000 mcs·s/m², or less). Exemplary ERG a-wave and b-wave amplitudes provided in the Example are from a mouse model of age-related retinal degeneration (e.g., dry AMD); ERG a-wave and b-wave responses characteristic of a healthy, at-risk, or disease eye in a human will be understood by a person of ordinary skill in the art.

In certain embodiments, a subject administered PBM according to the present disclosure does not develop AMD, or does not experience progression of AMD.

A “patient” or “subject” includes an animal, such as a human, dog, cat, monkey, ape, cow, horse, sheep, lamb, pig, mouse, rat, rabbit or guinea pig. The animal can be a mammal, such as a non-primate or a primate (e.g., monkey, ape, and human). In some embodiments, a subject is a human, such as a human infant, child, adolescent, or adult, such as an adult about 20, 30, 35, 40, 45, 50, 55, 60, 65, 70, 75, 80, 85, 90, 95, 100, 105, or 110 years of age, or more. In some embodiments, a subject is a human of 50, 55, 60, 65, 70, 75, 80, 85, 90, 95, 100, 105, or 110 years of age, or more.

### Photobiomodulation (PBM) and PBM Therapy Parameters

“Photobiomodulation,” also referred to as “PBM” herein, refers to the effect of visible light energy, typically of a wavelength from about 500 nm-1000 nm, to stimulate, suppress, or otherwise modulate a biological activity. PBM is distinguishable from other forms of light-based intervention, such as photoablating or photocoagulating lasers, in that it does not cause significant damage to (e.g., cauterize, ablate, coagulate, kill, or scar) to target cells or tissue. Without being bound by theory, PBM may act at the cellular level by activating mitochondrial respiratory chain components, resulting in stabilization of metabolic function. For example, it has been suggested that cytochrome C oxidase (CCO) is a key photoacceptor of light in the far red to near infrared spectral wavelength range. Grossman et al., Lasers. Surg. Med. 22:212-218 (1998); Kara et al., J. Photochem. Photobiol. B. 27:219-223 (1995); Karu and Kolyakov, Photomed. Laser Surg. 23:355-361 (2005); Kara et al., Lasers Surg. Med. 36:307-314 (2005); and Wong-Riley et al., J. Biol. Chem. 280:4761-4771 (2005).

Typically, PBM for treating ocular conditions involves delivering light energy from an external light-emitting source to the eye of a subject or patient, wherein the light energy comprises one or more PBM wavelengths and is delivered with sufficient intensity (e.g., power density or irradiance, which may be measured at a target tissue, or an emission source, or at any point therebetween. Standard measurements in this regard include J/cm² and mW/cm². Determination of an appropriate power output to deliver light of appropriate energy(ies) and wavelength(s) to a target of interest can be performed using calculations and methods described in, for example, PCT Publication No. WO 2016/040534A1, the calculations, PBM methods, and devices of which are incorporated herein by reference. In certain embodiments, a target of interest comprises retinal tissue in an eye of a subject.

Presently disclosed methods comprise use of light having a wavelength in a red wavelength range and/or light having a wavelength in a near-infrared (NIR) wavelength range.

In certain embodiments, a wavelength in a red wavelength range is in a wavelength range from 620 nm to 750 nm. In further embodiments, a wavelength in the red wavelength range is in a wavelength range from 630 nm to 740 nm. In further embodiments, a wavelength in the red wavelength range is in a wavelength range from 640 nm to 730 nm. In still further embodiments, a wavelength in the red wavelength range is in a wavelength range from 650 nm to 720 nm. In yet further embodiments, a wavelength in the red wavelength range is in a wavelength range from 660 nm to 710 nm. In further embodiments, a wavelength in a red wavelength range is 670±50 nm, 670±40 nm, 670±30 nm, 670±25 nm, 670±20 nm, 670±15 nm, 670±10 nm, or 670±5 nm. In certain embodiments, a wavelength in a red wavelength range is 670 nm. In further embodiments, a wavelength in a red wavelength range is 660±40 nm, 660±30 nm, 660±25 nm, 660±20 nm, 660±15 nm, 660±10 nm, or 660±5 nm. In certain embodiments, a wavelength in a red wavelength range is 670 nm, or is 660 nm.

In certain embodiments, a wavelength in a NIR wavelength range is in a wavelength range from 750 nm to 950 nm. In further embodiments, a wavelength in the NIR wavelength range is in a wavelength range from 800 nm to 900 nm. In still further embodiments, a wavelength in the NIR wavelength range is in a wavelength range from 825 nm to 875 nm.

In certain embodiments, a wavelength in a NIR wavelength range is 830±50 nm, 830±40 nm, 830±30 nm, 850±25 nm, 830±20 nm, 830±15 nm, 830±10 nm, or 830±5 nm, or about 830 nm. In certain embodiments, a wavelength in a NIR wavelength range is 830 nm.

In certain embodiments, a wavelength in a NIR wavelength range is 850±50 nm, 850±40 nm, 850±30 nm, 850±25 nm, 850±20 nm, 850±15 nm, 850±10 nm, or 850±5 nm, or about 850 nm. In certain embodiments, a wavelength in a NIR wavelength range is 850 nm.

In certain embodiments, a method further comprises use of (an effective amount of) PBM light comprising light having a wavelength in a yellow wavelength range. In some embodiments, a method comprises use of a combination of light having a wavelength in two or three wavelength ranges (e.g., any two, or all three of, light having a wavelength in a yellow wavelength range, light having a wavelength in a red wavelength range, and light having a wavelength in a near-infrared (NIR) wavelength range). In some embodiments, PBM therapy according the present disclosure includes light of one or more wavelength between 550 nanometers and 1060 nanometers. In certain embodiments, PBM therapy according the present disclosure includes light of one or more wavelengths between 550 nanometers and 980 nanometers.

In certain embodiments, a wavelength in a yellow wavelength range is in a wavelength range from 550 nm to 620 nm. In further embodiments, a wavelength in the yellow wavelength range is in a wavelength range from 560 nm to 610 nm. In still further embodiments, a wavelength in the yellow wavelength range is in a wavelength range from 570 nm to 600 nm. In particular embodiments, a wavelength in the yellow wavelength range is 590 nm±30 nm, 590 nm±25 nm, 590 nm±20 nm, 590 nm±15 nm, 590 nm±10 nm, or 590 nm±5 nm. In certain embodiments, a wavelength in the yellow wavelength range is 590 nm.

It will be understood that PBM light of different wavelengths (or in different wavelength ranges) can be administered simultaneously, sequentially, and/or contemporaneously). Accordingly, a method can comprise, for example, administering PBM light comprising light having a wavelength in a red wavelength range, and then administering PBM light comprising light having a wavelength in a yellow range. Alternatively, for example, a method can comprise simultaneous administration of PBM light comprising light having a wavelength in a red wavelength range, and PBM light comprising light having a wavelength in a yellow range.

Other parameters of PBM therapy according to the present disclosure include, for example: light emission; power density; pulsing or continuous light delivery; length of pulsed light; width of a pulsed light beam; temporal pulse shape(s), duty cycle(s), pulse frequency(ies); irradiance per pulse; beam diameter; sequence and number of exposures to the or more administered PBM lights or wavelengths; duration of a exposure to the one or more administered PBM lights or wavelengths; duration of a treatment session; whether a subject's eye is open or closed during all or part of a treatment; or the like.

For example, in certain embodiments, a PBM light or wavelength may be emitted at an intensity (e.g., power density) of from about 0.001 mW/cm² to about 100 mW/cm² or more; e.g., about 0.001, 0.005, 0.01, 0.05, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200 or more mW/cm², or any integer or non-integer value therewithin.

In some embodiments, any PBM light or wavelength of the present disclosure may have a fluence and/or an intensity at an emission surface of a light source in a range from about 0.1 nJ/cm² to about 50 J/cm², in a range from about 0.1 mJ/cm² to about 20 J/cm², in a range from about 0.1 mJ/cm² to about 10 J/cm², in a range from about 1 J/cm² to about 20 J/cm², in a range from about 1 J/cm2 to about 10 J/cm², or about 1, 1.5, 2, 2.5, 3, 3.5, 4, 4.5, 5, 5.5, 6, 6.5, 7, 7.5, 8, 8.5, 9, 9.5, 10, or more J/cm².

In embodiments comprising administration of PBM light of multiple wavelengths, two or more of the lights can be administered at the same or at different intensities.

In certain embodiments, for example, a method of the present disclosure comprises two or three of: light comprising a wavelength in the yellow wavelength range; light comprising a wavelength in the red wavelength range; and light comprising a wavelength in a NIR wavelength range, wherein: the light comprising the wavelength in the yellow wavelength range is emitted from a source at about 1, 2, 3, 4, 5, 6, 7, 8, 9, 10, 11, 12, 13, 14, 15, 16, 17, 18, 19, 20, 21, 22, 23, 24, or about 25 mW/cm², or more; the light comprising the wavelength in the red wavelength range is emitted from a source at about 1, 2, 3, 4, 5, 6, 7, 8, 9, 10, 15, 20, 25, 30, 35, 40, 45, 50, 55, 60, 65, 70, 75, 80, 85, or about 90 mW/cm2; the light comprising the wavelength in the NIR wavelength range is emitted from a source at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more mW/cm². In particular embodiments, a light comprising a wavelength in the yellow wavelength range (e.g., 590 nm) is emitted from a source at about 5 mW/cm². In particular embodiments, a light comprising a wavelength in the red wavelength range (e.g., 670 nm) is emitted from a source at about 6 mW/cm². In particular embodiments, a light comprising a wavelength in the NIR wavelength range (e.g., 850 nm) is emitted from a source at about 8 mW/cm². In certain embodiments, a method comprises administering a light comprising a wavelength in the yellow wavelength range (e.g., 590 nm), a light comprising a wavelength in the red wavelength range (e.g., 670 nm), and a light comprising a wavelength in the NIR wavelength range (e.g., 830 nm), wherein the light comprising the wavelength in the yellow wavelength range is emitted at about 5 mW/cm², the light comprising the wavelength in the red wavelength range is emitted at about 65 mW/cm², and the light comprising the wavelength in the NIR wavelength range is emitted at about 8 mW/cm². It will be understood that in any of the presently disclosed methods, a light that comprises a PBM wavelength in a particular wavelength range (e.g., yellow, red, or near infrared) or comprises a particular PBM wavelength or wavelength range (e.g., 590 nm, 670 nm, 830 nm, or 850 nm) can be partially, substantially, or entirely comprised of light of the PBM wavelength or wavelength range.

In other words, a recited PBM wavelength or wavelength range can account for, e.g., about 5%, 10%, 20%, 30%, 40%, 50%, 60%, 70%, 80%, 90%, 91%, 92%, 93%, 94%, 95%, 96%, 97%, 98%, 99%, or more of the light.

In any embodiment comprising use of multiple (i.e., two or more) wavelengths, any two or more of the multiple wavelengths can be emitted or delivered simultaneously, concurrently, or in any sequence, including overlapping and non-overlapping sequences and sequences wherein a resting period comprising no PBM light, or PBM light of a different wavelength, is interspersed between the emission or delivery of the two or more wavelengths.

In any of the embodiments disclosed herein, a PBM light can be delivered in a pulsed fashion, a continuous fashion, or both. Pulsed light can be delivered in any shape, frequency, irradiance, duty cycle, or other parameter appropriate to a treatment. In some embodiments, PBM light is pulsed at a frequency of about 1 Hz to 100 Hz, from about 100 Hz to about 1 kHz, less than 1 Hz, or more than 100 Hz. In embodiments that comprise two or more PBM wavelengths, any two or three wavelengths may be delivered to a subject or subject eye in a pulsed or a continuous fashion. In some embodiments, one of: a wavelength in the yellow wavelength range, a wavelength in the red wavelength range, and a wavelength in the NIR wavelength range are delivered at least in part in a pulsed fashion; and another of a wavelength in the yellow wavelength range, a wavelength in the red wavelength range, and a wavelength in the NIR wavelength range are delivered at least in part in a continuous fashion.

In certain embodiments, a PBM light according to the present disclosure has any beam diameter that is suitable to reach and sufficiently contact a target area (e.g., cell, organ, body party, or tissue). A beam diameter can be measured at a treatment pane, at the point of exit or emission from a light source, or at any point therebetween. Unless otherwise indicated, a diameter of a light beam as described herein refers to the diameter at a treatment plane. Suitable beam diameters and light intensities can be readily determined by a person of ordinary skill in the art in regard to, for example, the particular cell, organ, body part, or tissue to be targeted, the type, severity, and stage of disease or condition, the size, age, eye (iris) color of the subject, the distance from the point of light emission to the target cell, organ, body part, or tissue, or the like. For example, in certain embodiments, a beam for providing PBM light to a retinal tissue in an eye of a subject having or suspected of having dry age-related macular degeneration can be about 10, 15, 20, 30, 35, 40, 45, or more mm in diameter. In particular embodiments, a beam has a diameter of about 30 mm.

Treatment exposure times will also be readily determined by those of ordinary skill in the art with regard to, for example, the particular relevant feature(s) of the subject, the disease or condition to be treated, the type, intensity, and/or wavelength(s) of light being administered, or the like. In some embodiments, a treatment can comprise administering one or more light comprising a PBM wavelength for between about 0.0001 milliseconds and about 1 hour, or more. In certain embodiments, a treatment session comprises administering one or more PBM light, wherein each of the one or more light is administered for about 1, 5, 10, 15, 20, 25, 30, 35, 40, 45, 50, 55, 60, 65, 70, 75, 80, 85, 90, 95, 100, 105, 110, 115, 120, 130, 140, 150, 160, 170, or 180 seconds or more. In some embodiments, a total time of a treatment session comprises the combined duration of the light administration(s), and can wavelength range from less than about one minute to ten or more minutes. In particular embodiments, a treatment session comprises a total treatment (e.g., exposure) time of less than about 20 minutes, less than about 15 minutes, less than about 10 minutes, or less than about 9, 8, 7, 6, 5, 4, 3, or 2 minutes, or less than about one minute. In some embodiments, a treatment session comprises a total treatment (e.g., exposure) time of less than about 5 minutes, or is about 4 minutes. The total treatment time can refer to treatment of a single eye (even if more than one eye of a subject is to receive treatment in the session) or of more than one eye.

In certain embodiments, a method comprises administering PBM light on 1, 2, 3, 4, 5, 6, or 7 days in a one-week (7-day) period. In certain embodiments, a method comprises administering PBM light one or more times per week for 2 or more (e.g., 2, 3, 4, 5, 6, 7, 8, 9, 10, 11, 12, 13, 14, 15, 16, 17, 18, 19, or more consecutive weeks). In certain embodiments, a method comprises administering PBM light monthly, bimonthly, or once every three, four, five, six, seven, eight, nine, ten, eleven, or twelve months.

In any of the herein described embodiments, a method can comprise a treatment session that includes one or more phases, wherein the one or more phases are each characterized by any one or a combination of the herein described PBM parameters; e.g., light color, light wavelength, pulsed versus continuous light, light intensity, light delivered to an open eye, light delivered to a closed eye through an eyelid, light delivered to a first eye and optionally to a second eye, a resting period wherein no light or no PBM light is being administered or delivered, varying intensities of the light(s), or the like.

For example, in certain embodiments, a method can comprise a first phase comprising administering light (e.g., one or two or more beams) comprising a first and a second PBM wavelength, and a second phase comprising administering light comprising a third wavelength. It will be understood that phases can comprise lights, wavelengths, continuous versus pulsing, rest times, changes in intensity, or the like in any sequence and/or combination. In general, the lights, wavelengths, light intensities, treatment times, phases, beam diameters, pulsing and/or continuous delivery during a treatment session according to the presently disclosed methods will be selected according to their efficacy or potential efficacy for treating the given subject and/or condition, by the convenience and comfort threshold for the subject (e.g., not keeping an eye open for more than few minutes to receive PBM), and by safety considerations (e.g., using light of an appropriate intensity for PBM, and avoiding undesirable heating, ablation, cauterization, coagulation, or other forms of damage). Non-limiting examples include all of the PBM treatment parameters, and the specific treatment methods and combinations of the PBM treatment parameters described in PCT Patent Publication No. WO 2016/040534A1, which are incorporated herein by reference.

In specific embodiments, a method comprises administering PBM light for a about 180 or more seconds, wherein the method comprises one or more phases that each independently comprise a continuous administration of PBM, pulsed PBM, or both, and can include a wavelength in one, two, or three of a red wavelength range, a yellow wavelength range, and a near infrared wavelength range.

### Devices

Light suitable for PBM can be produced, for example, by a laser (e.g., a low-power laser) or a non-coherent light source (e.g., a light emitting diode (LED), a laser diode (e.g., a gallium-aluminum-arsenic (GaAlAs) laser diode, an aluminum gallium indium phosphide (AlGaLnP) laser diode, a diode-pumped solid state (DPSS) laser, a vertical cavity surface-emitting laser (VCSEL) diode, or the like), a lamp, or the like).

Any suitable light-emitting device can be used to provide PBM therapy of the present disclosure. It will be understood that PBM light can be generated and/or administered using a single device or source or using different devices or sources during a treatment session or over the course of a treatment regimen (e.g., comprising multiple treatment sessions). Exemplary devices include all of those disclosed in PCT Publication No. WO 2016/040534A1, as well as those comprising combinations of the features disclosed therein, (see also the VALEDA™ Light Delivery System by LumiThera) and in U.S. Pat. No. 9,592,404. Other devices include the Warp 10™ (Quantum Devices, Inc.; Barneveld, Wis.) and the GentleWaves® (Light Bioscience LLC; Virginia Beach, Va.) instruments. Accordingly, contemplated herein are devices including self-standing devices, wearable devices (e.g., in the form of glasses-like devices, which may be binocular or monocular, eye-patch-type devices, masks, or the like), hand-held devices, and the like.

In certain embodiments, a device comprises a microprocessor or microcontroller that modulates one or more parameter of PBM therapy; e.g., any one or more of the parameters described herein. In some embodiments, a device is programmable and can, for example, provide a PBM therapy that is customized or tailored for a particular subject, subject eye, or group, class, or category of subjects. Therapeutic settings (e.g., parameters) for providing PBM to a subject can be adjusted during a treatment session (e.g., in real time), or between treatment sessions, or over the course of a treatment regimen, regime, or program based on. Exemplary microprocessors and devices, systems, and methods comprising use of the same for providing PBM therapy are described in PCT Publication No. WO 2016/040534A1, and are incorporated herein.

Also provided are devices and systems useful for imaging, gathering or capturing data from, and/or processing information about an eye of a subject. Suitable devices include, for example, fundus cameras and related filters, ocular coherence tomography devices, ERG machines, dark field adaptometers (e.g., Labrique et al., BMC Opthnalmol. 15:74 (2015); AdaptDx®), and multimodal imaging devices (e.g., Spectralis® and related modules).

### PBM Based on Patient Criteria

In certain embodiments, presently disclosed methods are based, in part, on subject criteria that may indicate an improved likelihood of or capacity for response to PBM to prevent or treat the AMD.

Briefly, in the present disclosure, it was determined that PBM comprising light having a wavelength in a red wavelength range (e.g., 670 nm) and/or in a NIR wavelength range (e.g., 830 nm) can delay, prevent, attenuate, or otherwise treat dry AMD when administered an early stage or prior to an early stage of disease.

In particular, in certain embodiments, presently disclosed methods of preventing, delaying, or attenuating dry age-related macular degeneration or of preserving or improving a retinal function, such as a b-wave response (e.g., intensity thereof and/or duration thereof to light stimulus), an a-wave response (e.g., intensity thereof and/or duration thereof to light stimulus), or both, in an eye of a subject at risk for or exhibiting dry age-related retinal degeneration, comprise administering PBM light as disclosed herein when an eye of the subject exhibits any one or more of (a)-(p): (a) hypo-pigmentation of the retinal pigment epithelium (RPE); (b) hyper-pigmentation of the RPE; (c) mottling of the RPE; (d) vacuolation in the RPE and/or the sub-RPE space, optionally in the Bruch's membrane; (e) multivesicular bodies in the RPE and/or the sub-RPE space, optionally in the Bruch's membrane; (f) a region of partial or complete loss of RPE; (g) drusen or drusen-like deposits, optionally being dome-shaped and/or having hard borders and/or a whitish color; (h) accumulation of lipofuscin; (i) spontaneous choroidal neovascularization (CNV); (j) subretinal hemorrhage; (k) subretinal cellular infiltrates; (m) basal laminar deposits in the RPE; (n) a region of thickening of Bruch's membrane; (o) an ERG response amplitude of a b-wave that is reduced as compared to a response in a healthy eye (in certain embodiments, a b-wave amplitude of an at-risk or diseased eye is about 200 μV, about 150 μV, or less to a flash of an intensity that is about 25,000 mcs·s/m², 20,000 mcs·s/m², 15,000 mcs·s/m², 10,000 mcs·s/m², 5,000 mcs·s/m², or less); (p) an ERG response amplitude of an a-wave that is reduced as compared to a response in a healthy eye (in certain embodiments, an a-wave amplitude of an at-risk or diseased eye is about 150 μV, about 100 μV, or less to a flash of an intensity that is about 25,000 mcs·s/m², 20,000 mcs·s/m², 15,000 mcs·s/m², 10,000 mcs·s/m², 5,000 mcs·s/m², or less). Exemplary ERG a-wave and b-wave amplitudes provided in the Example are from a mouse model of age-related retinal degeneration (e.g., dry AMD); ERG a-wave and b-wave responses characteristic of a healthy, at-risk, or disease eye in a human will be understood by a person of ordinary skill in the art.

In certain embodiments, these phenotypes are representative of age-related retinal degeneration and dry AMD and have been described, e.g., in Zhao et al., PLoS One 6(4):e19456 (2011), the retinal degeneration markers, imaging techniques, and assays of which are incorporated herein by reference.

In certain embodiments, presence or absence of a herein-described phenotype or symptom (e.g., one or more of (a)-(p) above) can be determined using any technique known to a person of ordinary skill in the art; e.g., fundus photography, electroretinography, ocular coherence tomography, histopathology, or the like. See Zhao et al., PLoS One 6(4):e19456 (2011).

In certain embodiments, prior to administering the PBM light, the subject does not exhibit vision loss in the eye, wherein vision loss optionally comprises a blurred spot in a center of a field of vision.

In certain embodiments, prior to administering the PBM light, the eye of the subject does not exhibit medium-size drusen, large-size drusen, soft drusen, drusen having a soft border, and/or drusen having a yellow color.

In certain embodiments, prior to administering the PBM light, the eye of the subject does not exhibit geographic atrophy (GA) affecting a fovea.

In certain embodiments, prior to administering the PBM light, the subject does not report difficulty in: (i) seeing in a center of a field of vision; (ii) seeing in dim light; (iii) seeing and/or perceiving straight lines; and/or (iv) seeing and/or perceiving colors.

In some embodiments, prior to administering the PBM light, the eye had an AREDS categorization of AREDS 1 (i.e., no AMD). In some embodiments, prior to administering the PBM light, the eye had an AREDS categorization of AREDS 2 (i.e., early stage AMD). In some embodiments, prior to administering the PBM light, the eye had an AREDS categorization of AREDS 3 (i.e., intermediate AMD).

In some embodiments, a method further comprises, prior to and/or after administering the PBM light, performing one or more of an electroretinogram, fluoroscein angiography, ocular coherence tomography (OCT), spectral domain ocular coherence tomography, enhanced depth imaging OCT, swept source OCT, retinal oximetry, OCT angiography, dark adaptometry, an FST test, or fundus imaging, optionally comprising performing fundus autofluoresence, on the eye of the subject.

In some embodiments, a method further comprises, prior to administering the PBM light, identifying the subject as being at-risk for developing or progressing dry AMD.

### Example

### Animal Study of PBM Therapy for Dry Age-Related Macular Degeneration

### Study Design

To study the effects of PBM therapy in the red and NIR wavelength ranges protects on retinal dysfunction in AMD, the following experiments were conducted in 38 to 48-week old Nrf2^{-/-} mice, an art-accepted model for age-related ocular pathologies including AMD (see, e.g., Zhao et al., PLoS One 6(4):e19456 (2011)). Nrf2^{-/-} mice develop age-dependent degenerative pathology in the retinal pigment epithelium (RPE). Drusen-like deposits, accumulation of lipofuscin, spontaneous choroidal neovascularization (CNV) and sub-RPE deposition of inflammatory proteins develop in Nrf2^{-/-} mice by or after approximately 12 months. Autophagy-related vacuoles and multivesicular bodies develop within the RPE and in Bruch's membrane of aged Nrf2^{-/-} mice.

In the present study, experiments were conducted in accordance with the ARVO statement for the Use of Animals in Ophthalmic and Vision Research and all animal protocols approved by the UWM IACUC. Mice were housed in dim cyclic light conditions (12 h light, 12 h dark) with food and water available ad libitum. Animals were divided into sham and PBM treatment groups. Mice were placed in a plexiglass box and treated once-per-day for 5 days per week for 12 weeks with 670 nm or 830 nm LED arrays (Quantum Devices, Inc. Barneveld, Wis.) at a dose of 4.5 J/cm² (25 mW/cm² for 180 sec). Sham-treated animals were restrained, but not exposed to 670 nm or 830 nm light.

Electroretinogram (ERG) recordings were obtained prior to treatment (baseline) and following PBM or sham treatment (HMsERG OcuScience Inc., Henderson, Nev.) A scotopic intensity series (100 mcd·s/m² to 25000 mcd·s/m²) was recorded.

In particular, baseline ERG recordings were obtained prior to treatment. Following PBM or sham treatments, mice were anesthetized and retinal function assessed by full-field flash-evoked electroretinography (ERG). Mice were dark-adapted overnight and prepared under red dim light. Animals were anesthetized with isoflurane and placed on a heating pad at 37° C. during recordings. Pupils were dilated with 0.1% atropine and mild topical anesthesia (proparacaine 0.5%). Full-field ERGs were obtained in a Ganzfeld dome (76 mm diameter Flash Dome with 55 mm aperture) using nylon-coated gold thread electrode placed on the corneal surface, overlaid with 1% methylcellulose and a contact lens. A sub dermal needle reference electrode and a ground needle electrode were placed in the cheek and tail, respectively. A high-intensity flash unit (HMsERG instrument, Ocuscience Inc., NV) was used to provide flash intensities (ranging from 10 mcd·s/m² to 25000 cd·s/m²) for scotopic intensity series response measurements.

Following in vivo assessments, mice were euthanized and retinal tissues prepared for biochemical and histological analysis.

Results

ERG is a diagnostic test that measures the electrical activity generated by neural and non-neuronal cells in the retina in response to a light stimulus. The electrical response is a result of a retinal potential generated by light-induced changes in the flux of transretinal ions. ERG has been used to provide important diagnostic information on a variety of retinal disorders and also used to monitor disease progression.

Briefly, and without wishing to be bound by theory, the a-wave is the initial corneal-negative deflection, derived from the cones and rods of the outer photoreceptor layers. This wave is thought to reflect the hyperpolarization of the photoreceptors due to closure of sodium ion channels in the outer-segment membrane. Absorption of light is believed to trigger rhodopsin to activate transducin, a G-protein. This is thought to lead to the activation of cyclic guanosine monophosphate phosphodiesterase (cGMP PDE), eventually leading to a reduction in the level of cGMP within the photoreceptor. This, in turn, is believed to leads to closure of the sodium ion channels, resulting in a decrease of inwardly directed sodium ions, or a hyperpolarization of the cell. The a-wave amplitude is measured from baseline to the trough of the a-wave.

Without wishing to be bound by theory, the b-wave is corneal-positive deflection and is derived from the inner retina, predominantly Muller and ON-bipolar cells. Hyperpolarization of the photoreceptor cells results in a decrease in the amount of neurotransmitter released, which subsequently leads to a depolarization of the post-synaptic bipolar cells. Bipolar-cell depolarization increases the level of extracellular potassium, subsequently generating a transretinal current. The transretinal current is thought to depolarize the radially oriented Muller cells and generate corneal-positive deflection. The b-wave amplitude is generally measured from the trough of the a-wave to the peak of the b-wave. The b-wave is the most common component of the ERG used in clinical and experimental analysis of human retinal function.

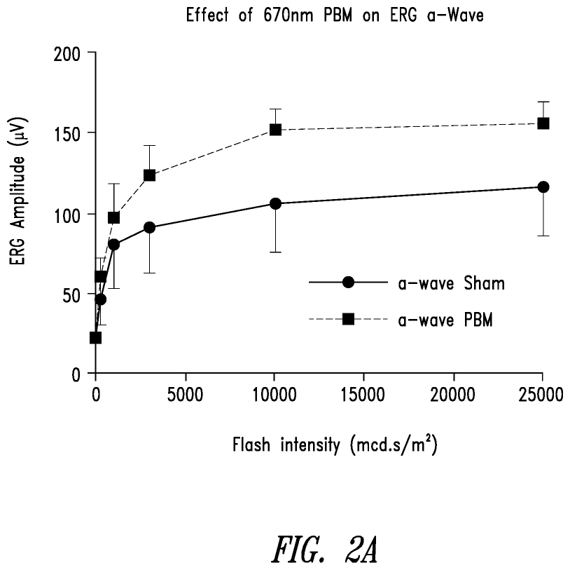

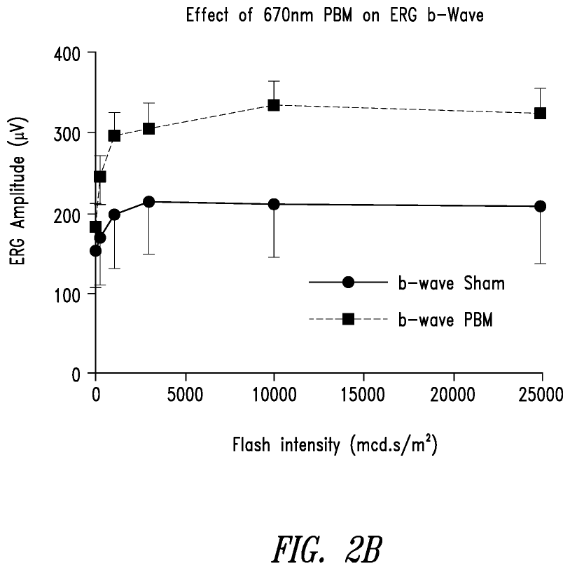

In the experiment testing the effect of 670 nm PBM, ERG a-wave responses increased from an amplitude of 80 μV at a flash intensity of 100 mcd·s/m² to 100 μV at a flash intensity of 25,000 mcd·s/m² in sham-treated mice, compared to 100 μV and 150 μV in 670 nm PBM-treated mice, representing an increase of 1.5 fold in the treated mice (FIG. 2A). The b-wave showed similar differences; in the sham-treated mice, amplitude increased from 150 to 200 μV, and from 200 to 350 μV in the 670 nm treated animals, representing a 1.8-fold improvement following PBM treatment (FIG. 2B).

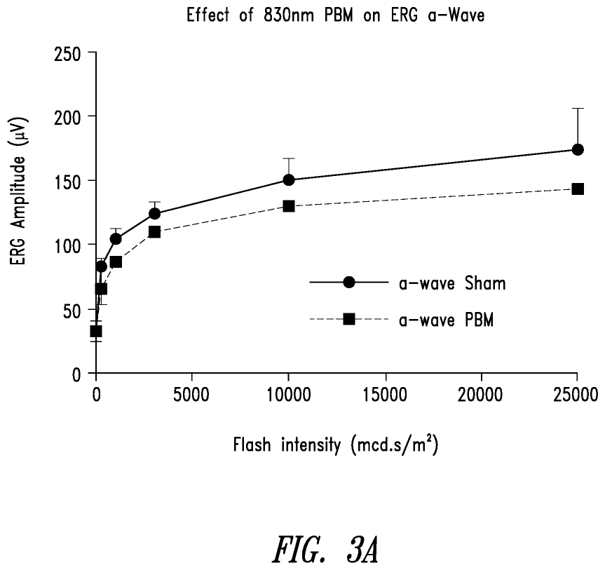

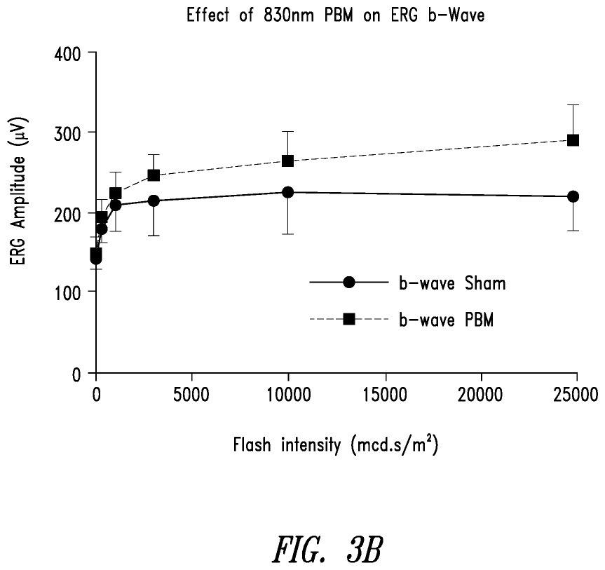

In the experiment testing the effect of 830 nm PBM, ERG a-wave responses were greater in sham-treated than in PBM-treated mice. ERG a-wave amplitude increased from 80 μV at 100 mcd·s/m² to 150 mV at 25,000 mcd·s/m² in sham-treated mice, compared to 80 μV to 100 μV in 830 nm PBM-treated mice (FIG. 3A). In contrast, the b-wave in sham-treated mice did not increase above 200 μV with increasing light intensity; however, in the 830 nm treated animals, the b-wave increased from 200 to 300 μV, representing a 1.5 fold improvement. The retinoprotective effect of PBM was most evident in the b-wave of the ERG, which is the most common component of the ERG used in clinical and experimental analysis of human retinal function.

These data show that PBM therapy comprising 670 nm light has a significant protective effect on retinal function in Nrf2^{-/-} mice, and also show that 830 nm PBM has a protective effect.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for preventing or delaying onset and/or progression of dry age-related macular degeneration (dry AMD) in an eye of a subject, the method comprising administering to the eye an effective amount of photobiomodulation (PBM) light comprising:
(i) light having a wavelength in a red wavelength range; and/or
(ii) light having a wavelength in a near-infrared (NIR) wavelength range, when the eye of the subject exhibits any one or more of (a)-(p):
(a) hypo-pigmentation of the retinal pigment epithelium (RPE);
(b) hyper-pigmentation of the RPE;
(c) mottling of the RPE;
(d) vacuolation in the RPE and/or the sub-RPE space;
(e) multivesicular bodies in the RPE and/or the sub-RPE space;
(h) a region of partial or complete loss of RPE;
(g) drusen or drusen-like deposits;
(h) accumulation of lipofuscin;
(i) spontaneous choroidal neovascularization (CNV);
(j) subretinal hemorrhage;
(k) subretinal cellular infiltrates;
(m) basal laminar deposits in the RPE;
(n) a region of thickening of Bruch's membrane;
(o) an ERG response amplitude of a b-wave that is reduced as compared to a ERG response amplitude of a b-wave in a healthy eye;
(p) an ERG response amplitude of an a-wave that is reduced as compared to a ERG response amplitude of an a-wave in a healthy eye.

2. A method for preserving or improving a retinal function in an eye of a subject, the method comprising administering to the eye an effective amount of photobiomodulation (PBM) light comprising:
(i) light having a wavelength in a red wavelength range; and/or
(ii) light having a wavelength in a near-infrared (NIR) wavelength range, when the eye of the subject exhibits any one or more of (a)-(p):
(a) hypo-pigmentation of the retinal pigment epithelium (RPE);
(b) hyper-pigmentation of the RPE;
(c) mottling of the RPE;
(d) vacuolation in the RPE and/or the sub-RPE space, optionally in the Bruch's membrane;
(e) multivesicular bodies in the RPE and/or the sub-RPE space, optionally in the Bruch's membrane;
(f) a region of partial or complete loss of RPE;
(g) drusen or drusen-like deposits, optionally being of small size, being dome-shaped, having hard borders, and/or having a whitish color;
(h) accumulation of lipofuscin;
(i) spontaneous choroidal neovascularization (CNV);
(j) subretinal hemorrhage;
(k) subretinal cellular infiltrates;
(m) basal laminar deposits in the RPE;
(n) a region of thickening of Bruch's membrane;
(o) an ERG response amplitude of a b-wave that is reduced as compared to a ERG response amplitude of a b-wave in a healthy eye;
(p) an ERG response amplitude of an a-wave that is reduced as compared to a ERG response amplitude of an a-wave in a healthy eye.

3. The method of claim 1, wherein prior to administering the PBM light, the subject does not exhibit vision loss in the eye.

4. The method of claim 1, wherein prior to administering the PBM light, the eye of the subject does not exhibit medium-size drusen, large-size drusen, soft drusen, drusen having a soft border, and/or drusen having a yellow color.

5. The method of claim 1, wherein prior to administering the PBM light, the eye of the subject does not exhibit geographic atrophy (GA) affecting a fovea.

6. The method of claim 1, wherein prior to administering the PBM light, the subject does not report difficulty in:
(i) seeing in a center of a field of vision;
(ii) seeing in dim light;
(iii) seeing and/or perceiving straight lines; and/or
(iv) seeing and/or perceiving colors.

7. The method of claim 1, wherein prior to administering the PBM light, the eye had an AREDS categorization of AREDS 1.

8. The method of claim 1, wherein prior to administering the PBM light, the eye had an AREDS categorization of AREDS 2.

9. The method of claim 1, wherein prior to administering the PBM, the eye had an AREDS categorization of AREDS 3.

10. The method of claim 1, wherein the wavelength in a red wavelength range is 670±50 nm or 660±40 nm.

11. The method of claim 1, wherein the wavelength in a NIR wavelength range is 830 nm±50 nm or 850 nm±50 nm.

12. The method of claim 1, wherein the light of (i) and/or the light of (ii) is administered to the eye at a dose of at least about 4.5 J/cm².

13. The method of any claim 1, wherein the method comprises administering the PBM light on 1, 2, 3, 4, 5, 6, or 7 days in a week.

14. The method of claim 13, comprising administering the PBM light for 2, 3, 4, 5, 6, 7, 8, 9, 10, 11, 12, or more consecutive weeks, wherein each of the consecutive weeks independently comprises administering the PBM light for 1, 2, 3, 4, 5, 6, or 7 days.

15. The method of claim 1, further comprising administering an effective amount of PBM light comprising light having a wavelength in the yellow wavelength range.

16. The method of claim 15, wherein the wavelength in the yellow wavelength range is in a wavelength range from 550 nm to 620 nm.

17. The method of claim 16, wherein the wavelength in the yellow wavelength range is in a wavelength range from 560 nm to 610 nm.

18. The method of claim 16, wherein the wavelength in the yellow wavelength range is in a wavelength range from 570 nm to 600 nm.

19. The method of claim 16, wherein the wavelength in the yellow wavelength range is 590 nm±15 nm.

20. The method of claim 1, further comprising, prior to and/or after administering the PBM light, performing one or more of an electroretinogram, fluoroscein angiography, ocular coherence tomography (OCT), spectral domain ocular coherence tomography, enhanced depth imaging OCT, swept source OCT, retinal oximetry, OCT angiography, dark adaptometry, an FST test, or fundus imaging, optionally comprising performing fundus autofluoresence, on the eye of the subject.
